(19) United States
(12) Patent Application Publication
(10) Pub. No.: US 2006/0184022 A1
(43) Pub. Date: Aug. 17, 2006

(54) Ultrasound for the diagnosis and treatment of carpal tunnel and other localized nerve compression conditions

(76) Inventor: Lanny L. Johnson, Okemos, MI (US)

Correspondence Address: Wilson D. Swayze, Jr., 3804 Clearwater Ct., Plano, TX, 75025, US

(21) Appl. No.: 11/336,458

(22) Filed: Jan. 20, 2006

Related U.S. Application Data
(60) Provisional application No. 60/647,556, filed on Jan. 27, 2005.

(51) Int. Cl. A61B 8/00; A61B 8/12; A61B 8/14

(52) U.S. Cl. 600/437; 600/438; 600/439; 600/440

(57) ABSTRACT

An apparatus and methods for diagnosing and treating nerve compression conditions in a patient, such as carpal tunnel syndrome, tarsal tunnel syndrome and other nerve compression conditions. Ultrasound transducer heads having an array of high frequency piezoelectric crystal elements mounted in the transducer head capable of delivering frequencies of 7.5 MHz or greater, and a low frequency piezocomposite element mounted in the transducer head capable of delivering frequencies of 0.25 MHz to 1.5 MHz at an intensity of about 0.25 W/cm² to 2.0 W/cm² can be used to diagnose and also treat the conditions. If necessary, the high frequency piezoelectric elements can be used to visualize the region of therapy to more accurately direct the low frequency ultrasound energy.

### PRIORITY AND CROSS-REFERENCE TO RELATED APPLICATIONS

The present application claims priority under 35 USC section 119 and application Ser. No. 60/647,556 filed on Jan. 27, 2005.

### STATEMENT REGARDING FEDERALLY SPONSORED RESEARCH OR DEVELOPMENT

Not Applicable

### BACKGROUND OF THE INVENTION

(1) Field of the Invention

The present invention relates generally to carpal tunnel syndrome and other localized nerve compression conditions. Specifically, the present invention relates to the diagnosis and treatment of nerve compression conditions using ultrasound. The present invention relates to an apparatus and methods for diagnostics and therapeutic treatment of nerve compression conditions.

(2) Description of the Related Art

Nerve compression conditions are common problems which arise in the body when there is pressure on a peripheral nerve or nerve root which leads to ischemia. Edema surrounding the point of applied pressure may develop. The ischemia decreases the nerve's conduction abilities. These conditions sometimes lead to fibrosis and possibly to the loss of motor endplates which will lead to muscle atrophy. Severe chronic conditions, such as carpal tunnel syndrome, results in atrophy which leads to a clawlike deformity of the hand. Nerve compression can be caused by a variety of factors, including vascular, inflammatory, trauma, anatomical, metabolic, lactrognic and neoplastic pathologies. Many peripheral nerves have been associated with nerve compression conditions, and more than one type of condition can be found in relation to specific nerves. Carpal tunnel syndrome, anterior interosseous syndrome and pronator syndrome are due to pressure on the median nerve. Cubital and ulnar tunnel syndromes are caused by compression of the ulnar nerve at the elbow. Radial tunnel syndrome, posterior interosseous syndrome, and superficial radial nerve syndrome (Wartenberg syndrome) are caused by compression of the radial nerve. Seventh cranial/facial nerve outlet demyelination, caused by pulsing of vessel against nerve at the cranial outlet. In the lower body, tarsal tunnel syndrome is caused by compression of the posterior tibial nerve, and meralgia paresthetica is due to pressure on the lateral cutaneous nerve of the thigh. Electromyelograms (EMG) and nerve conduction tests are electrodiagnostic methods currently used to confirm the diagnosis of a the nerve compression conditions. Once a diagnosis has been made, surgical decompression is still a therapy of choice to alleviate the pressure upon the nerves, however only a small percentage of patients require surgery.

Acoustic energy has been applied in medical settings both diagnostically and therapeutically. U.S. Patent Application Publication No. 2002/0133094 A1 to Wilcox et al. describes an apparatus and methods for the diagnosis of stress and injury in soft tissue by detecting the response of the soft tissue to acoustic energy. The use of ultra high-frequency acoustic energy is now routinely used diagnostically as a imaging technique which is referred to as sonography. Sonography is capable of imaging fine superficial structures such as tendons and nerves in the region of interest in nerve compression conditions. Ultrasound at lower frequencies than those used during sonography has been used therapeutically. U.S. Patent Application Publication No. 2003/0153849 A1 to Huckle et al. discloses methods and an apparatus for the purpose of treating connective tissues and increasing vascularization utilizing ultrasound. U.S. Pat. No. 5,558,092 to Unger et al. disclose methods and apparatus for performing diagnostic ultrasound simultaneously with the application of therapeutic ultrasonic waves. The apparatus is used to perform imaging of a region of a patient while simultaneously applying therapeutic ultrasound for the purpose of rupturing vesicles administered to the patient. The vesicles can contain bioactive agents which are released when ruptured.

While the related art teach diagnostic ultrasonography and ultrasound therapy, there still exists a need for improved tools which are capable of delivering ultrasound for both diagnostic and therapeutic purposes for nerve compression conditions.

### OBJECTS

Therefore, it is an object of the present invention to provide an apparatus for the diagnosis and treatment of nerve compression conditions.

It is further an object of the present invention to provide a methods of diagnosing and treating nerve compression conditions with the apparatus.

These and other objects will become increasingly apparent by reference to the following description.

### SUMMARY OF THE INVENTION

The present invention provides an apparatus for the diagnosis and treatment of nerve compression conditions of a patient comprising: (a) an ultrasound transducer head having at least one aperture; (b) a high frequency piezoelectric crystal array mounted in the transducer head capable of delivering frequencies of 7.5 MHz or greater mounted for Doppler imaging, to provide excellent near field resolution for viewing an inflamed nerve to assist in the diagnosis of the nerve compression condition of the patient; and (c) a low frequency piezocomposite element mounted in the transducer head capable of delivering frequencies of 0.25 MHz to 1.5 MHz at an intensity of about 0.25 W/cm² to 2.0 W/cm² so as to provide therapeutic ultrasound energy to the inflamed nerve causing the nerve compression condition so as to treat the patient.

In further embodiments of the apparatus, the Doppler technique is continuous wave Doppler ultrasound, pulsed Doppler ultrasound, color Doppler sonography, or power Doppler sonography. In still further embodiments, the array of high frequency piezoelectric crystal elements is a linear array or a phased array. In further embodiments, the therapeutic ultrasound energy is focused by means of an acoustic lens or electronic focusing. In still further embodiments the low frequency piezoelectric crystal element comprises a 1-3 piezocomposite array. In still further embodiments, the low frequency piezoelectric crystal element comprises a piezoelectric fiber composite.

The present invention provides a method of diagnosing and treating a nerve of a patient with a nerve compression condition comprising: (a) providing an ultrasound system comprising an ultrasound transducer head having at least one aperture; a high frequency piezoelectric crystal array mounted in the transducer head capable of delivering frequencies of 7.5 MHz or greater mounted for Doppler imaging, to provide excellent near field resolution for viewing an inflamed nerve to assist in the diagnosis of the nerve compression condition of the patient; and a low frequency piezocomposite element mounted in the transducer head capable of delivering frequencies of 0.25 MHz to 1.5 MHz at an intensity of about 0.25 W/cm to 2.0 W/cm² so as to provide therapeutic ultrasound energy;(b) measuring a cross-sectional area of the nerve with the array of high frequency piezoelectric crystal elements of the ultrasound system; (c) comparing the measured cross-sectional area of the nerve to an average cross sectional area of the nerve in a population of healthy individuals to provide a comparative value; (d) diagnosing whether a nerve compression condition exists based upon the comparative value; (e) if a diagnosis of a nerve compression condition is made, applying ultrasound energy generated from the one or more low frequency piezoelectric crystal elements of the ultrasound system to the target nerve with the ultrasound transducer head as a treatment for the diagnosed nerve compression condition.

In further embodiments of the method the nerve is a median nerve. In still further embodiments of the method when the nerve is a median nerve, the mean cross-sectional area of the nerve in a population of healthy individuals is 10 mm². In further embodiments the nerve cross-sectional area is measured using a frequency of 7.5 MHz. In still further embodiments, the therapeutic ultrasound comprises a frequency of about 1 MHz to 2.25 MHz. In still further embodiments the therapeutic ultrasound is applied at an intensity of 0.25-1.5 W/cm². In still further embodiments, the therapeutic ultrasound is applied at an intensity of 1.0 W/cm². In still further embodiments the therapeutic ultrasound is applied at a pulse mode of 1:4. In further embodiments the therapeutic ultrasound is applied for a duration of fifteen minutes per session. In further embodiments the therapeutic ultrasound is applied in one or more sessions. In still further embodiments the therapy is applied daily.

### BRIEF DESCRIPTION OF THE DRAWINGS

FIG. 1 shows one embodiment of an ultrasound transducer head 10 of the present invention showing the high frequency piezoelectric array 20 and low frequency piezocomposite 30.

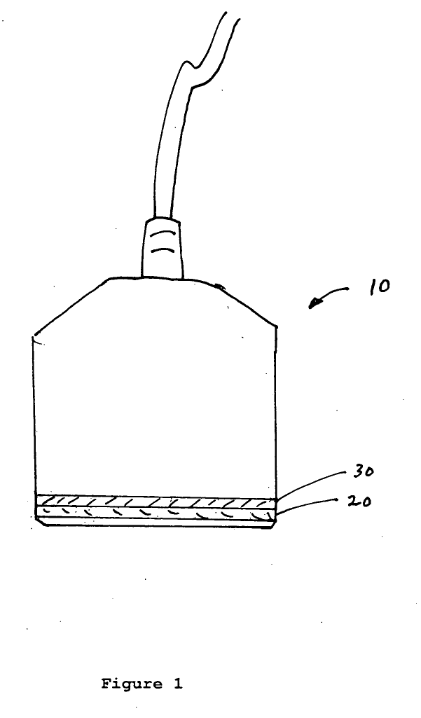

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “nerve compression condition” used herein refers to median nerve trauma, entrapment, or inflammation neuropathies such as carpal tunnel syndrome and related conditions such as anterior interosseous syndrome and pronator syndrome due to pressure on the median nerve. The term also encompasses peripheral nerve or nerve root: cubital and ulnar tunnel syndromes caused by compression of the ulnar nerve at the elbow; radial tunnel syndrome, posterior interosseous syndrome, and superficial radial nerve syndrome (Wartenberg syndrome) caused by compression of the radial nerve; tarsal tunnel syndrome caused by compression of the posterior tibial nerve, and meralgia paresthetica is due to pressure on the lateral cutaneous nerve of the thigh. Finally, the term applies to compression conditions of nerves of the cervical or lumbar vertebrae such as sciatic, peroneal, posterior tibial, femoral, saphenous, obturator, lateral femoral cutaneous or related nerves.

The term “Doppler imaging” used herein refers to continuous wave Doppler ultrasound, pulsed Doppler ultrasound, color Doppler sonography, and power Doppler sonography. Power Doppler sonography also refers to amplitude Doppler sonography, color Doppler energy (CDE), color amplitude imaging (CAI), and ultrasound angiography.

Carpal tunnel syndrome (CTS) is the most common entrapment neuropathy which manifests as paresthesia of the fingers and thumb weakness. The carpal tunnel is a tight canal through which both the median nerve and tendons pass between the forearm and the hand. The median nerve is compressed if the tendons in the carpal tunnel become inflamed, which leads to the tingling, numbness, shooting pain in the wrist or forearm, and weakness in grip. Similar anatomy in the ankle and foot results in a similar, although less common condition termed tarsal tunnel syndrome (TTS).

Diagnostic Ultrasound: Nerve conduction velocity tests are traditionally used to diagnose carpal tunnel syndrome and electromyograms are performed to exclude other conditions which may appear. The article by Ellis et al., Imaging 14:217-222 (2002), shows that high resolution ultrasound can image nerve courses and internal structure. Also, it is useful to identify sites and causes of nerve entrapment syndromes. Recently, quantitative ultrasonography has been shown to be useful as a tool in the diagnosis of patients with carpal tunnel syndrome (El Miedany et al., Rheumatology 43(7): 887-895). El Miedany et al. found that there is a high degree of correlation between conduction velocity abnormalities detected by traditional nerve conduction diagnostic tests and cross-sectional area of the median nerve. It was found that the upper limit for normal values of the mean cross-sectional area of the median nerve was 10 mm². The ultrasonography is highly accurate when used diagnostically and locates the cause of the nerve compression condition.

Therapeutic Ultrasound: Ultrasound treatment within the range of 0.5-2.0 W/cm² has the potential to induce biophysical effects within tissue (Ebenbichler et al. British Medical Journal 1998 Mar. 7; 316: 731-735). Ebenbichler et al showed improvements in carpal tunnel syndrome in subjective symptoms and electroneurographic variables, such as nerve conduction velocity in patients treated for 15 minute sessions with a 1 MHz frequency, 1.0 W/cm² intensity ultrasound procedure with a pulse mode of 1:4. Multiple sessions lasting for 15 minutes were repeated over a number of weeks. Mourad et al. (Neurosurgery May 2001; 48(5): 1136-1140) were successful in treating rats with nerve compression injuries using a time-averaged intensity of 0.25 W/cm2 at a frequency of 2.25 MHz for a one minute application for three days a week. Paik et al. (Muscle Nerve September 2002; 26(3): 356-361) showed significant improvements in compound action potential amplitude (CMAP) in rabbits with median nerve compression conditions following ultrasound treatment with an intensity of 1.5 W/cm².

Piezoelectric arrays: Piezoelectric arrays are traditionally used in diagnostic ultrasonography. They consist of one or more piezoelectric elements sandwiched between backing/matching layers. Lead zirconate titanate (PZT) and polyvinylidene difluoride (PVDF) are two piezoelectric materials are often used for ultrasound transducers.

Piezocomposite materials: Piezocomposites are a form of piezoceramic material surrounded by piezoelectrically passive polymer which have excellent performance. A preferred piezocomposite is the 1-3 piezocomposite. 1-3 piezocomposite materials are piezoelectric ceramics combined with non-piezoelectric polymers which are arrays of piezoceramic rods in a polymer matrix. Another piezocomposite is the 2-2 composite, which is an array of alternating piezoceramic strips. The number designations refer to the mechanical connectivity of the active and inactive phases. Injection molded piezocomposite materials can be made having resonant frequencies between 50 kHz and 1 MHz. Methods for making such piezoelectric ceramic/polymer composite transducers are described in U.S. Pat. No. 5,340,510 to Bowen. Piezocomposite materials and arrays are described in U.S. Pat. No. 6,806,622 to Schmidt et al., and U.S. Pat. No. 6,277,299 to Seyed-Bolorforosh, each of these are hereby incorporated herein by reference in their entirety.

Piezoelectric fiber composites have fiber piezoelectric elements embedded in epoxy in a random pattern. Piezoelectric fiber composites are suitable for resonant frequencies between 40 kHz and 6 MHz, whereas monoliths (bulk PZT or lead-zirconate-titanate, ceramics) are recommended outside this range. Piezoelectric fiber composites can be machined into various shapes including concave surfaces.

Ultrasound Transducer Heads: The preferred transducer heads of the present invention comprise an array of high frequency piezoelectric crystal elements mounted in the transducer head capable of delivering frequencies of 7.5 MHz or greater, and a low frequency piezocomposite element mounted in the transducer head capable of delivering frequencies of 0.25 MHz to 1.5 MHz at an intensity of about 0.25 W/cm² to 2.0 W/cm². The high frequency array of the transducer head can be used for diagnostic purposes and can be used to as a means to visualize and direct the low frequency ultrasound energy to the proper location. The low frequency piezocomposites can be shaped to focus the beam.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

I claim:

1. An apparatus for the diagnosis and treatment of nerve compression conditions of a patient comprising:
(a) an ultrasound transducer head having at least one aperture;
(b) a high frequency piezoelectric crystal array mounted in the transducer head capable of delivering frequencies of 7.5 MHz or greater mounted for Doppler imaging, to provide excellent near field resolution for viewing an inflamed nerve to assist in the diagnosis of the nerve compression condition of the patient; and
(c) a low frequency piezocomposite element mounted in the transducer head capable of delivering frequencies of 0.25 MHz to 1.5 MHz at an intensity of about 0.25 W/cm² to 2.0 W/cm² so as to provide therapeutic ultrasound energy to the inflamed nerve causing the nerve compression condition so as to treat the patient.

2. The apparatus of claim 1 wherein the Doppler technique is continuous wave Doppler ultrasound, pulsed Doppler ultrasound, color Doppler sonography, or power Doppler sonography.

3. The apparatus of claim 1 wherein the array of high frequency piezoelectric crystal elements is a linear array or a phased array.

4. The apparatus of claim 1 wherein the therapeutic ultrasound energy is focused by means of an acoustic lens or electronic focusing.

5. The apparatus of claim 1 wherein the low frequency piezoelectric crystal element comprises a 1-3 piezocomposite array.

6. The apparatus of claim 1 wherein the low frequency piezoelectric crystal element comprises a piezoelectric fiber composite.

7. A method of diagnosing and treating a nerve of a patient with a nerve compression condition comprising:
(a) providing an ultrasound system comprising an ultrasound transducer head having at least one aperture; a high frequency piezoelectric crystal array mounted in the transducer head capable of delivering frequencies of 7.5 MHz or greater mounted for Doppler imaging, to provide excellent near field resolution for viewing an inflamed nerve to assist in the diagnosis of the nerve compression condition of the patient; and a low frequency piezocomposite element mounted in the transducer head capable of delivering frequencies of 0.25 MHz to 1.5 MHz at an intensity of about 0.25 W/cm² to 2.0 W/cm² so as to provide therapeutic ultrasound energy;
(b) measuring a cross-sectional area of the nerve with the array of high frequency piezoelectric crystal elements of the ultrasound system;
(c) comparing the measured cross-sectional area of the nerve to a mean cross-sectional area of the nerve in a population of healthy individuals to provide a comparative value;
(d) diagnosing whether a nerve compression condition exists based upon the comparative value;
(e) if a diagnosis of a nerve compression condition is made, applying ultrasound energy generated from the one or more low frequency piezoelectric crystal elements of the ultrasound system to the target nerve with the ultrasound transducer head as a treatment for the diagnosed nerve compression condition.

8. The method of claim 7 wherein the nerve is a median nerve.

9. The method of claim 8 wherein the mean cross-sectional area of the nerve in a population of healthy individuals is 10 mm².

10. The method of claim 7 wherein the nerve cross-sectional area is measured using a frequency of 7.5 MHz.

11. The method of claim 7 wherein the therapeutic ultrasound comprises a frequency of about 1 MHz to 2.25 MHz.

12. The method of claim 7 wherein the therapeutic ultrasound is applied at an intensity of 0.25-1.5 W/cm².

13. The method of claim 7 wherein the therapeutic ultrasound is applied at an intensity of 1.0 W/cm².

14. The method of claim 7 wherein the therapeutic ultrasound is applied at a pulse mode of 1:4.

15. The method of claim 7 wherein the therapeutic ultrasound is applied for a duration of fifteen minutes per session.

16. The method of claim 15 wherein the therapeutic ultrasound is applied in one or more sessions.

17. The method of claim 16 wherein the therapy is applied daily.
